(19) United States
(12) Patent Application Publication
(10) Pub. No.: US 2012/0129183 A1
(43) Pub. Date: May 24, 2012

(54) METHODS AND KITS FOR ISOLATING PLACENTAL DERIVED MICROPARTICLES AND USE OF SAME FOR DIAGNOSIS OF FETAL DISORDERS

(75) Inventors: Anat Aharon, Kiryat-Tivon (IL); Benjamin Brenner, Haifa (IL)

(73) Assignees: RAPPAPORT FAMILY INSTITUTE FOR RESEARCH IN THE MEDICAL SCIENCES, Haifa (IL); FUND FOR MEDICAL RESEARCH DEVELOPMENT OF INFRASTRUCTURE AND HEALTH SERVICES, Haifa (IL)

(21) Appl. No.: 13/379,379

(22) PCT Filed: Jun. 24, 2010
(86) PCT No.: PCT/IL2010/000504

§ 371 Date: Feb. 9, 2012

Related U.S. Application Data
(60) Provisional application No. 61/219,824, filed on Jun. 24, 2009.

(51) Int. Cl. C12Q 1/68; G01N 33/53; G01N 33/566

(52) U.S. Cl. 435/6.12; 436/501; 435/7.8

(57) ABSTRACT

A prenatal method of analyzing a fetus is disclosed. The method comprising: (a) isolating placental derived microparticles; and (b) analyzing at least one component of the contents of the placental derived microparticles, wherein the at least one component is indicative of a characteristic of the fetus.

### FIELD AND BACKGROUND OF THE INVENTION

The present invention, in some embodiments thereof, relates to a method and kit for isolating placental derived microparticles from a maternal blood sample and to the use of same for fetal profiling.

Prenatal screening to detect potential birth defects (such as Down syndrome, chromosome abnormalities, genetic diseases and other conditions) is commonly carried out during pregnancy. Screening is preferably performed during early stages of pregnancy. Syndromes caused by an extra or missing chromosome (aneuploidy) are among the most widely recognized genetic disorders in humans and are currently being tested using procedures such as amniocentesis and chorionic villus sampling (CVS). However, although efficient in predicting chromosomal aberrations, the amniocentesis or CVS procedures carry a 0.5-1% or 2-4% of procedure related risks for miscarriage, respectively.

Microvesicles (MVs), which include microparticles and exosomes, are found in blood circulation in normal physiologic conditions and are increased in a variety of diseases. Microparticles (MPs) are membrane vesicles that shed from various cellular surfaces and contain a small amount of cell cytoplasm material. Cellular microparticles are formed by cytoskeleton structural rearrangements and vary in size (from about 0.1 to 1 μm) and in phospholipids and protein compositions. MPs bear DNA and RNA [Reich C F et al. Exp Cell Res. (2009) 10; 315:760-8] and expose membrane antigens that are specific for the cells from which they are derived [Diamant et al., Eur J Clin Invest (2004) 34:392-401]. For example, in the circulation there are MPs that were shed from platelets, from endothelial cells or from leukocytes. In cancer patients tumor cell-derived MPs can be detected and placental-derived MPs are found in pregnant women.

There are two mechanisms that can result in microparticle formation—cell apoptosis or activation—after exposure to cytokines or toxins and in a variety of pathologies (such as inflammation, cancer, diabetes, and other vascular disease). In the blood, MPs appear to be a major source of RNA with the membrane structure shielding the nucleic acids from digestion by blood nucleases. Moreover, circulating microparticles modulate target cells and facilitate cell-to-cell interactions by transferring proteins and RNA (e.g. microRNA) between cells, thereby elevating protein expression on the target cell membranes and inducing cell signaling.

Circulating nucleic acids can provide markers of both diagnostic and prognostic significance. MPs in the blood can contain mRNA from their origin cells, such as tumor, in a form that can be analyzed by genomic techniques. In pregnancies, extracellular mRNA provides a source of material for assessing fetal gene expression [Ng E K et al., Proc Natl Acad Sci USA. (2003) 15; 100].

The trophoblast cells, which begin as the outer covering of early fetus blastocyte, provide the route of nourishment between the maternal endometrium and the developing embryo. Human villous trophoblast (HVT) cells covering the placental vili provide the surface for exchange of oxygen and nutrients with maternal circulation and they are the only cells with embryonic phenotype which are exposed to the maternal circulation. Placental trophoblast differentiation is accompanied by apoptosis and results in release of syncytiotrophoblast MPs into the maternal circulation.

The syncytiotrophoblast-derived MPs are associated with circulatory fetal nucleic acids in-vitro [DNA and mRNA, Gupta A K et al., Clinical Chemistry (2004) 50: 2187-2190]. Syncytiotrophoblast-derived MPs may be detected in maternal circulation beginning from the second trimester (i.e. using ELISA and anti-NDOG2 antibodies), their numbers increase during the third trimester and they participate in systemic inflammatory responses in normal or preeclamptic pregnancies [Germain S J et al., J. Immunol. (2007) 178: 5949-56]. MPs of placental origin were labeled with an anti-NDOG1 antibody and evaluated by flow cytometry [Aharon A et al. J Thromb Haemost. 2009 Mar. 13].

Previous studies describe fetal analysis by isolating fetal nucleated cells (e.g. erythrocytes) from the maternal blood and subjecting them to genetic analysis (see for example U.S. Pat. No. 5,750,339).

U.S. Patent Application No. 20080261822 describes methods for prenatal diagnosis by in situ staining of trophoblast cells. According to their teachings, transcervical specimens are collected from a pregnant subject and are subjected to trophoblast-cell specific immuno-staining followed by in situ DNA-based genetic analysis in order to determine fetal gender and/or identify chromosomal and/or DNA abnormalities in a fetus.

Additional art includes Orozco A F et al., American Journal of Pathology. (2008) 173:1595-1608.

### SUMMARY OF THE INVENTION

According to an aspect of some embodiments of the present invention there is provided a prenatal method of analyzing a fetus, the method comprising: (a) isolating placental derived microparticles; and (b) analyzing at least one component of the contents of the placental derived microparticles, wherein the at least one component is indicative of a characteristic of the fetus.

According to an aspect of some embodiments of the present invention there is provided a method of isolating placental derived microparticles from a blood sample obtained from a pregnant subject, the method comprising: (a) contacting the blood sample with at least one agent which specifically binds the placental derived microparticles and not to maternal microparticles under conditions that allow binding of the at least one agent to the placental derived microparticles; and (b) isolating the placental derived microparticles, thereby isolating the placental derived microparticles from the blood sample.

According to an aspect of some embodiments of the present invention there is provided an isolated population of microparticles comprising at least 80% placental derived microparticles, obtained according to the method of claim 3.

According to an aspect of some embodiments of the present invention there is provided a kit for prenatally analyzing a fetus, the kit comprising a packaging material packaging a first agent capable of specifically binding placental derived microparticles and a second agent for analyzing at least one component of the contents of the placental derived microparticles and instructions for use.

According to some embodiments of the invention, the method further comprises isolating the component from the placental derived microparticles following step (a) and prior to step (b).

According to some embodiments of the invention, the isolating is not effected by FACS.

According to some embodiments of the invention, the isolating is effected by immunoprecipitation.

According to some embodiments of the invention, the method further comprises centrifuging the blood sample as to obtain poor platelet plasma (PPP) prior to the contacting.

According to some embodiments of the invention, the agent comprises an antibody.

According to some embodiments of the invention, the antibody binds to a membrane polypeptide of the placental derived microparticles.

According to some embodiments of the invention, the antibody comprises an anti-NDOG1 antibody.

According to some embodiments of the invention, the agent binds a polypeptide selected from the group consisting of a human chorionic gonadotropin (HCG), a human Placental Lactogen (hPL), a NDOG1, a NDOG2, a NDOG5, a Trop-1 and a Trop-2.

According to some embodiments of the invention, the isolating is effected according to the method of claim 3.

According to some embodiments of the invention, the at least one component comprises a nucleic acid.

According to some embodiments of the invention, the at least one component comprises a polypeptide.

According to some embodiments of the invention, the characteristic is a fetal disorder.

According to some embodiments of the invention, the fetal disorder comprises a fetal chromosomal aberration.

According to some embodiments of the invention, the chromosomal aberration comprises an aneuploidy.

According to some embodiments of the invention, the fetal disorder comprises a fetal genetic mutation.

According to some embodiments of the invention, the genetic mutation comprises polymorphism of the 5,10-methylenetetrahydrofolate reductase (MTHFR) gene.

According to some embodiments of the invention, the characteristic is a sex of the fetus.

According to some embodiments of the invention, the placental derived microparticles are in a blood sample obtained from a pregnant subject.

According to some embodiments of the invention, the first agent comprises an antibody.

According to some embodiments of the invention, the antibody comprises an anti-NDOG1 antibody.

According to some embodiments of the invention, the at least one component is selected from the group consisting of a nucleic acid and a polypeptide.

According to some embodiments of the invention, the kit further comprises at least one agent for isolating nucleic acids from the placental derived microparticles.

According to some embodiments of the invention, the kit further comprises at least one for isolating polypeptides from the placental derived microparticles.

According to some embodiments of the invention, the second agent is selected from the group consisting of an oligonucleotide, a probe, an antibody and a dye.

According to some embodiments of the invention, the blood sample is selected from the group consisting of a whole blood, a fractionated whole blood, a diluted blood sample, an undiluted blood sample, a blood plasma, a blood serum and microparticles.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B are pictures showing the specificity of the trophoblast-cell specific antibody NDOG1. Placental human villous trophoblasts (HVT) were obtained from 24 week pregnant women, labeled with either isotype control IgG-PE or anti NDOG1-PE and analyzed by FACS. FIG. 1A illustrates HVT labeled with isotype control IgG-PE; and FIG. 1B illustrates HVT labeled with anti-NDOG1-PE.

FIG. 2 is a graph showing placental derived microparticles (MPs). MPs isolated from poor platelet plasma (PPP) of non pregnant women (NP), healthy pregnant women (HP) and women with gestational vascular complications (GVC), were labeled with anti-NDOG1 and evaluated by FACS.

FIGS. 3A-E are graphs showing elevation in placental MP levels in early stages of pregnancy. MPs were isolated from poor platelet plasma (PPP) of non-pregnant women (NP) and healthy pregnant women at different stages of pregnancy (gestational weeks 11, 13, 15 and 19 of pregnancy). The red area represents negative control IgG. The black curve represents percentage of MPs labeled with the placental marker anti-NDOG1.

FIGS. 4A-D are graphs showing separation of placental MPs from total MPs. MPs of 15 week pregnant women were labeled with the placental marker NDOG1 or with the maternal platelet marker CD41 prior to immunoseparation (FIGS. 4A-B) and after separation (FIGS. 4C-D).

FIG. 5 is a graph depicting microparticle-derived DNA concentration and quality. MPs were isolated from poor platelet plasma (PPP) of 19 week pregnant woman by ultracentrifugation. DNA was extracted by purification kit and DNA concentration and quality was evaluated by Nanodrop.

FIG. 6 is a graph depicting genetic profiling of trophoblast-derived microparticles using QF-PCR. Trophoblast cells were grown in-vitro, starved for 48 hours and supernatants were collected. Trophoblast MPs were isolated from the supernatants by serial centrifugations. DNA was extracted from the trophoblast MPs and molecular analysis was carried out using QF-PCR analysis for chromosomes 13, 18, 21, X and Y.

FIG. 7 is a graph depicting genetic profiling for methylenetetrahydrofolate reductase (MTHFR) polymorphism in placental-derived microparticles isolated from plasma of three different pregnant women evaluated by Rotor-gene PCR. Line 1 (blue line) is a control DNA sample with a normal MTHFR gene expression; Line 2 (yellow line) is a DNA sample with a MTHFR(C677T) mutation—heterozygote; Line 3 (purple line) is a DNA sample obtained from placental derived-MPs of pregnant woman 1 (at 21 weeks of gestation)—the fetus was found to be normal for MTHFR gene expression; Line 4 (turquoise line) is a DNA sample obtained from placental derived-MPs of pregnant woman 2 (at 20 weeks of gestation)—the fetus was found to harbor a MTHFR mutation (heterozygote); Line 5 (black line) is a DNA sample obtained from placental derived-MPs of pregnant woman 3 (at 20 weeks of gestation)—the fetus was found to harbor a MTHFR mutation (homozygote); and Line 6 (red line) is a H₂O sample.

FIG. 8 is a flow chart summarizing fetal genetic diagnosis (e.g. detection of fetal chromosomal aneuploidy).

### DESCRIPTION OF SPECIFIC EMBODIMENTS OF THE INVENTION

The present invention, in some embodiments thereof, relates to a method and kit for isolating placental, derived microparticles from a maternal blood sample and to the use of same for fetal profiling.

The principles and operation of the present invention may be better understood with reference to the drawings and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

It is known that the syncytiotrophoblast-derived microparticles (MPs) are associated with circulatory fetal nucleic acids in-vitro [DNA and mRNA, Gupta A K et al., Clinical Chemistry (2004) 50: 2187-2190]. However, until presently, it was not known that placental derived microparticles may be isolated from maternal blood in such a fashion that they may be used for genetic evaluation of the fetus.

As is shown hereinbelow and in the Examples section which follows, the present inventors have uncovered that placental derived microparticles may be isolated from maternal blood samples using an antibody which specifically binds a trophoblast specific protein, NDOG1 (see Example 4). The placental derived microparticles may then be used to extract nucleic acids therefrom (see Example 5) and profiled for fetal genetic characteristics including chromosomal aberrations (see Example 6) and genetic mutations (see Example 7). Furthermore, the present inventors have shown that placental derived microparticles are evident in the maternal blood from early stages of pregnancy (e.g. from at least gestational week 11, see Example 3) and therefore may be used for fetal diagnosis from early stages of pregnancy.

Thus, according to one aspect of the present invention there is provided a prenatal method of analyzing a fetus, the method comprising: (a) isolating placental derived microparticles; and (b) analyzing at least one component of the contents of the placental derived microparticles, wherein the at least one component is indicative of a characteristic of the fetus.

The term “prenatal” as used herein refers to any stage of a pregnancy occurring or existing before the birth of an offspring. According to the present teachings, the pregnant subject is a human female.

The term “fetus” as used herein refers to an unborn offspring at any stage of gestation beginning from fertilization, including an embryo or fetus, until birth.

The analysis may be effected at any stage of the pregnancy. According to one embodiment, the analysis is effected at gestational week 10, 11, 12, 13, 14, 15, 16, 17, 18, 19, 20, 21 or later.

It will be appreciated that the determination of the exact week of gestation during a pregnancy is well within the capabilities of one of ordinary skill in the art of Gynecology and Obstetrics.

The term fetus, as used herein refers to a healthy fetus or to a diseased fetus (e.g. carrying a genetic disease or mutation).

As used herein, the phrase “placental derived microparticles” refers to acellular particles comprising placental material that are between about 100 nm to about 10 μM or between about 100 nm to about 1.5 μM in diameter. According to one embodiment the microparticles are derived from the syncytiotrophoblast see Rusterholz et al., Fetal Diagn Ther. (2007) 22(4):313-7. Epub 2007 Mar. 15; or apoptotic bodies, see Hasselmann et al., Clin Chem (2001) 47:1488-1489). These microparticles are usually formed as a result of shedding (such as following cell activation, complement activity) and/or cell lysis (such as resulting from apoptosis) of the fetal placenta.

In order to analyze the fetus, placental derived microparticles are first isolated from a maternal blood sample. The blood sample may comprise whole blood, fractionated whole blood, diluted blood sample, undiluted blood sample, blood plasma, blood serum or microparticles.

As used herein, the term “isolating” refers to a physical isolation of placenta derived microparticles from the blood sample. Any isolation method known in the art may be used for isolation of the placenta derived microparticles, as described in further detail hereinbelow. According to one embodiment, the isolating is performed such that intact cells are not present in the sample with the particles.

According to one embodiment, methods are used to enrich for placental derived microparticles in the blood, prior to isolation. For example, the blood may be treated to remove platelets and other cells to obtain Poor-Platelet Plasma (PPP). This may be effected using techniques such as high spin centrifugation, as described in detail in the materials and methods section below.

It will be appreciated that maternal microparticles also exist within the blood sample (e.g. platelet derived microparticles, endothelial cell derived microparticles, leukocyte derived microparticles and erythrocyte derived microparticles) and therefore placental derived microparticles should be isolated using an agent which is capable of distinguishing between the two. Such an agent may include an antibody which specifically binds to a polypeptide expressed on the outer membrane of the placental derived microparticles. Alternatively, the agent may comprise a small permeable agent (e.g. antibody) which passes the microparticle membrane and binds to a polypeptide expressed inside the placental derived microparticles. Preferably, the agent of the present invention binds with at least 2.5 fold, more preferably at least 5 fold, more preferably at least 10 fold higher affinity to placental derived microparticles than to maternal microparticles.

Accordingly, the antibody may bind to any placental or trophoblast specific antigenic markers e.g. Trop-1, Trop-2, NDOG1, NDOG2, NDOG5, human chorionic gonadotropin (HCG), human Placental Lactogen (hPL), present on the surface or within the placental derived microparticles.

According to a specific embodiment of the present invention, the antibody is an anti-NDOG1 antibody (available, for example, from Serotec, Abcam, GenWay Biotech, Inc. and LifeSpan BioSciences).

Examples of antibodies which may be used to specifically bind placental derived microparticles include, but are not limited to, antibodies directed against trophoblast specific antigens such as HLA-G antibody, which is directed against part of the non-classical class I major histocompatibility complex (MHC) antigen specific to extravillous trophoblast cells (Loke, Y. W. et al., 1997. Tissue Antigens 50: 135-146); the anti human placental alkaline phosphatase (PLAP) antibody which is specific to the syncytiotrophoblast and/or cytotrophoblast (Leitner, K. et al., 2001, J. Histochemistry and Cytochemistry, 49: 1155-1164); the CHL1 (CD146) antibody which is directed against the melanoma cell adhesion molecule (MCAM) (Higuchi T., et al., 2003, Mol. Hum. Reprod. 9: 359-366); the CRL2 antibody which is directed against laeverin, a nowi protein that belongs to membrane-bound gluzincin metallopeptidases and expressed on trophoblasts (Fujiwara H., et al., 2004, Biochem. Biophys. Res. 313: 962-968); the H315 antibody which interacts with a human trophoblast membrane glycoprotein present on the surface of fetal cells (Covone A E and Johnson P M, 1986, Hum. Genet. 72: 172-173); the FT1.41.1 antibody which is specific for syncytiotrophoblasts and the 103 antibody (Rodeck, C., et al., 1995. Prenat. Diag. 15: 933-942), the NDOG5 antibody which is specific for extravillous cytotrophoblasts (Miller D., et al. 1999, Supra); the BC1 antibody (Bulmer, J. N. et al., Prenat. Diagn. 1995, 15: 1143-1153); the AB-154 or AB-340 antibodies which are specific to syncytio- and cytotrophoblasts or syncytiotrophoblasts, respectively (Durrant L et al., 1994, Prenat. Diagn. 14: 131-140); the glucose transporter protein (Glut)-12 antibody which is specific to syncytiotrophoblasts and extravillous trophoblasts during the 10th and 12th week of gestation (Gude N M et al., 2003. Placenta 24:566-570); the Mab FDO202N directed against the human placental lactogen hormone (hPLH) which is expressed by extravillous trophoblasts (Latham S E, et al., Prenat Diagn. 1996; 16(9):813-21).

Antibodies against other proteins which are expressed on trophoblast cells can also be used along with the present invention. Examples include, but are not limited to, the HLA-C which is expressed on the surface of normal trophoblast cells (King A, et al., 2000, Placenta 21: 376-87; Hammer A, et al., 1997, Am. J. Reprod. Immunol. 37: 161-71), the JunD and Fra2 proteins (members of the API transcription factor) which are expressed on extravillous trophoblasts (Bamberger A M, et al., 2004, Mol. Hum. Reprod. 10: 223-8), the nucleoside diphosphate kinase A (NDPK-A) protein which is encoded by the nm23-H1 gene and is expressed in extravillous trophoblasts during the first trimester (Okamoto T, et al., 2002, Arch. Gynecol. Obstet. 266: 1-4), Tapasin (Copeman J, et al., 2000, Biol. Reprod. 62: 1543-50), the CAR protein (coxsackie virus and adenovirus receptor) which is expressed in invasive or extravillous trophoblasts but not in villous trophoblasts (Koi H, et al., 2001, Biol. Reprod. 64: 1001-9), the human Achaete Scute Homologue-2 (HASH2) protein which is expressed in extravillous trophoblasts (Alders M, et al., 1997, Hum. Mol. Genet. 6: 859-67; Guillemot F, et al., 1995, Nat. Genet. 9: 235-42), the human chorion gonadotropin alpha (alpha HCG) which is expressed in trophoblasts (Schueler P A, et al., 2001, Placenta 22: 702-15), the insulin-like growth factor-II (IGF-II), the placental protein 5 (PP5) which is identical to tissue factor pathway inhibitor-2 (TFPI-2) and is expressed by cytotrophoblasts (Rube F et al., Biol Reprod. 2003; 68: 1888-94) and the placenta-specific genes (PLAC1, PLAC8 and PLAC9) which are exclusively expressed by cells of the trophoblastic lineage (Pant M et al., Mol Reprod Dev. 2002; 63: 430-6; Galaviz-Hernandez C, et al., 2003, Gene 309: 81-9; Cocchia M, et al., 2000, Genomics 68: 305-12).

After the agent binds the placental derived microparticles, the particles may be separated from the blood sample and/or from other microparticles by any method known to one of ordinary skill in the art such as by immunoprecipitation, by magnetic beads (e.g. Bioadamt beads) or by fluorescence activated cell sorting (FACS).

FACS analysis enables the detection of antigens present on cell or microparticle membranes such as e.g. NDOG1. Briefly, antigen specific antibodies (e.g. anti-NDOG1 antibodies) are linked to fluorophores and detection is performed by means of a cell sorting machine which reads the wavelength of light emitted from each cell or microparticle as it passes through a light beam. This method may employ two or more antibodies simultaneously. The FACS machine also enables to sort out cells or microparticles which specifically bind a specific antibody.

A multitude of flow cytometers are commercially available including for e.g. Becton Dickinson FACS can and FACScalibur (BD Biosciences, Mountain View, Calif.). Antibodies that may be used for FACS analysis are taught in Schlossman S, Boumell L, et al, [Leucocyte Typing V. New York: Oxford University Press; 1995] and are widely commercially available.

Immunoprecipitation (IP) enables the detection of antigens present on cell or microparticle membranes such as e.g. NDOG1. Briefly, the antibody (e.g. anti-NDOG1 antibody) may directly interact with a sample (e.g., blood sample, plasma sample etc.) and the formed complex can be further detected using a secondary antibody conjugated to beads (e.g., if the anti-NDOG1 antibody is a mouse monoclonal antibody, the secondary antibody may be an anti-mouse antibody conjugated to e.g., Sepharose beads or to magnetic beads such as Bioadamt beads). The beads can then be precipitated by centrifugation (for Sepharose beads) and separated from the sample using a magnetic column (for magnetic beads) or using an elution buffer.

According to an embodiment of the present invention, the isolated population of microparticles comprises at least about 10%, 20%, 30%, 40%, 50%, 60%, 70%, 80%, 90%, 95% or 100% placental derived microparticles.

In order to determine a characteristic of the fetus, the contents of the isolated placental derived microparticles are analyzed. Particular components of the contents include for example, fetal chromosomes, nucleic acids, polypeptides, endosomes and exosomes.

As used herein the phrase “analyzing” refers to classifying a characteristic, a disease, disorder or a symptom, determining predisposition to a disease or syndrome or a severity of a disease or syndrome or forecasting an outcome of a disease or syndrome and/or prospects of recovery. The term “detecting” may also optionally encompass any of the above.

As used herein the term “characteristic” refers to any distinctive trait of the fetus including, for example, gender, hair color, skin color, eye color, or any other hereditary trait which may be determined by fetal genetic testing. Furthermore, the term characteristic may also refer to paternal testing of the fetus as to determine the biological parents thereof.

According to the present teachings, analyzing a fetus may be carried out in order to determine if the fetus has genetic disorders or mutations and has a likelihood of birth defects. Birth defects which may be analyzed according to the present teachings include, but are not limited to, neural tube defects, spina bifida, cleft palate, metabolic diseases, neural tube defects, sickle cell anemia, hemophilia, thalassemia (e.g. Beta-thalassemia), chromosome abnormalities or aberrations including e.g. common translocations (e.g., Robertsonian translocation), chromosomal deletions and/or microdeletions (e.g., Angelman syndrome, DiGeorge syndrome), chromosomal anueploidy (e.g., Down syndrome), single gene disorders (e.g., cystic fibrosis, Tay-Sachs disease, Canavan disease, Gaucher disease, Familial Dysautonomia, Niemann-Pick disease, Fanconi anemia, Ataxia telaugiestasia, Bloom syndrome, Familial Mediterranean fever (FMF), X-linked spondyloepiphyseal dysplasia tarda, factor XI), DNA-methylation related disorders [e.g., imprinting disorders such as Angelman Syndrome, Prader-Willi Syndrome, Beckwith-Wiedemann syndrome, Myoclonus-dystonia syndrome (MDS)], as well as disorders which are caused by minor chromosomal aberrations (e.g., minor trisomy mosaicisms, duplication sub-telomeric regions, interstitial deletions or duplications) as described in further detail below.

It will be appreciated that the present invention enables fetal analysis in a non-invasive fashion. However, the present teachings may be combined with other prenatal testing procedures including amniocentesis, chorionic villius sampling, ultrasonography (e.g. nuchal translucency ultrasound), serum marker testing or genetic screening.

Analyzing a characteristic of a fetus according to the present invention can be effected by determining a level (amount) of a component comprised inside placental derived microparticles, wherein the level is correlated with predisposition to, presence or absence of a characteristic or a disease, staging of a disease and the like.

The level of these components may be up-regulated or down-regulated compared to those found in a similar sample obtained from a healthy fetus (i.e. control data).

According to the present teachings, a change in one component (e.g. in a chromosome) may be indicative of a characteristic of the fetus (e.g. genetic disorder). Thus, chromosomal abnormality or aberration may refer to an abnormal number of chromosomes (e.g., trisomy 21, monosomy X) or to chromosomal structure abnormalities (e.g., deletions, translocations, etc).

For example, a deletion of part of the short arm of chromosome 5 is indicative of Cri du chat syndrome; an extra copy of chromosome 21 (trisomy 21) is indicative of Down syndrome; a trisomy of chromosome 18 is indicative of Edwards syndrome; extra genetic material of chromosome 15 is indicative of Isodicentric 15 (also called IDIC (15), Inverted duplication 15, extra Marker, Inv dup 15, partial tetrasomy 15); a partial deletion of the short arm of chromosome 4 is indicative of Wolf-Hirschhorn syndrome; a deletion in terminal 11q is indicative of Jacobsen syndrome; an extra chromosome X in male fetuses (XXY) is indicative of Klinefelter's syndrome; an extra chromosome X in female fetuses is indicative of Triple-X syndrome (XXX); a trisomy of chromosome 13 is indicative of Patau Syndrome (also called D-Syndrome or trisomy-13); a missing sex chromosome (X instead of XX or XY) is indicative of Turner syndrome; an extra chromosome Y in male fetuses is indicative of (XYY syndrome); an extra 47th autosomal chromosome which can originate from any of the 24 different human chromosomes leads to an extra genetic material [called a small supernumerary marker chromosome (sSMC)] can be indicative of Cat-eye syndrome, Idic15 (described above) and Pallister-Killian syndrome.

According to another embodiment, analyzing a characteristic of a fetus according to the present invention can be effected by analyzing a sequence of a polynucleotide or a polypeptide comprised in placental derived microparticles obtained from the maternal blood sample, wherein the sequence is correlated with predisposition to, presence or absence of a characteristic or a disease, staging of a disease and the like.

For example, Gaucher's disease may be diagnosed in fetuses by sequencing of the beta-glucosidase gene or by analyzing Gaucher-causing mutations e.g. Type I (N370S homozygote), Type II (1 or 2 alleles L444P) and Type III (1-2 copies of L444P); Beta-thalassemia (β-thalassemia) may be diagnosed in fetuses by sequencing of the HBB gene on chromosome 11; Bloom syndrome (BLM, also known as Bloom-Torre-Machacek syndrome) may be diagnosed in fetuses by sequencing for mutations in the BLM gene; increased predisposition to breast cancer may be diagnosed in fetuses by sequencing of either of two genes on chromosomes 17 (BRCA1) and 13 (BRCA2); Canavan disease, also called Canavan-Van Bogaert-Bertrand disease, may be diagnosed in fetuses by testing for aspartoacylase deficiency or aminoacylase 2 deficiency; Cystic Fibrosis (also known as CF) may be diagnosed in fetuses by analysis for mutations in the CFTR gene (on chromosome 7); Fabry disease (also known as Fabry's disease, Anderson-Fabry disease, angiokeratoma corporis diffusum and alpha-galactosidase A deficiency) may be diagnosed in fetuses by analysis for mutations in the GLA gene; Fanconi anemia may be diagnosed in fetuses by analysis for mutations in the following genes: FANCA, FANCB, FANCC, FANCD1, FANCD2, FANCE, FANCF, FANCG, FANCI, FANCJ, FANCL, FANCM and FANCN; Familial dysautonomia (FD, also called Riley-Day syndrome) may be diagnosed in fetuses by analysis for mutations in the IKBKAP gene on chromosome 9; Familial Mediterranean Fever (FMF) may be diagnosed in fetuses by analysis for mutations in the MEFV gene located on the short arm of chromosome 16 (16p13); Glucose-6-phosphate dehydrogenase deficiency may be diagnosed in fetuses by analysis for mutations on band Xq28 of the X chromosome; Maple syrup urine disease may be diagnosed in fetuses by analysis for mutations in the following genes: BCKDHA, BCKDHB, DBT and DLD; Mucolipidosis type IV (ML IV) may be diagnosed in fetuses by analysis for mutations in the MCOLN1 gene; Niemann-Pick disease may be diagnosed in fetuses by analysis for mutations in the SMPD1 gene (diagnosis for Niemann-Pick disease types A and B) and mutations in NPC1 and NPC2 (diagnosis for Niemann-Pick disease, type C(NPC)); Tay Sach's disease may be diagnosed in fetuses by analysis for genetic mutation on the HEXA gene on chromosome 15 and neural tube defects may be diagnosed in fetuses by analysis for homozygosity for the T allele of the C677T polymorphism in the gene encoding the folate dependent enzyme 5,10-methylenetetrahydrofolate reductase (MTHFR).

Control data may be obtained from the literature or by analyzing the placental microparticles of a fetus known to be healthy (using other diagnostic techniques, such as the ones described herein above).

Thus, according to the present teachings, analyzing the contents of the placental derived microparticles is effected by first isolating the contents from the microparticles.

Methods of isolating DNA or RNA are well known in the art, such as those described herein below.

For example, DNA purification may be carried out by methods involving cell lysis, protein extraction, and DNA precipitation using 2 to 3 volumes of 100% ethanol, rinsing in 70% ethanol, pelleting, drying, and resuspension in water or any other suitable buffer (e.g., Tris-EDTA). Preferably, following such a procedure, DNA concentration is determined, such as by measuring the optical density (OD) of the sample at 260 nm (wherein 1 unit OD=50 μg/ml DNA). Alternatively, DNA can be obtained by adding a protein digestion enzyme (e.g., proteinase K), followed by denaturation (e.g., boiling at 95° C. for 5-10 minutes).

RNA purification may be carried out by, for example, phenol-chloroform extraction using for example TRI Reagent, TRIzol or Trisure (available e.g. from Sigma-Aldrich, Invitrogen or Moline). Purification of short (less than 200 nucleotides) RNA species, such as siRNA, miRNA and tRNA may also be carried out for fetal analysis.

It will be appreciated that the present teachings contemplate purification and analysis of fragmented nucleic acid sequences or intact nucleic acid sequences.

The presence and/or level of a specific nucleic acid sequence can be determined using an isolated polynucleotide (e.g., a polynucleotide probe, an oligonucleotide probe/primer) capable of hybridizing to a fetal nucleic acid sequence or a portion thereof. Such a polynucleotide can be at any size, such as a short polynucleotide (e.g., of 15-200 bases), and intermediate polynucleotide (e.g., 200-2000 bases) or a long polynucleotide larger of 2000 bases.

The isolated polynucleotide probe used by the present invention can be any directly or indirectly labeled RNA molecule (e.g., RNA oligonucleotide, an in vitro transcribed RNA molecule), DNA molecule (e.g., oligonucleotide, cDNA molecule, genomic molecule) and/or an analogue thereof [e.g., peptide nucleic acid (PNA)] which is specific to the fetal transcript of the present invention.

The term “oligonucleotide” refers to a single stranded or double stranded oligomer or polymer of ribonucleic acid (RNA) or deoxyribonucleic acid (DNA) or mimetics thereof. This term includes oligonucleotides composed of naturally-occurring bases, sugars and covalent internucleoside linkages (e.g., backbone) as well as oligonucleotides having non-naturally-occurring portions which function similarly to respective naturally-occurring portions.

Oligonucleotides designed according to the teachings of the present invention can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988) and “Oligonucleotide Synthesis” Gait, M. J., ed. (1984) utilizing solid phase chemistry, e.g. cyanoethyl phosphoramidite followed by deprotection, desalting and purification by for example, an automated trityl-on method or HPLC.

The oligonucleotide of the present invention is of at least 17, at least 18, at least 19, at least 20, at least 22, at least 25, at least 30 or at least 40, bases specifically hybridizable with sequence alterations described hereinabove.

The isolated polynucleotide used by the present invention can be labeled either directly or indirectly using a tag or label molecule. Such labels can be, for example, fluorescent molecules (e.g., fluorescein or Texas Red), radioactive molecule (e.g., ³²P-γ-ATP or ³²P-α-ATP) and chromogenic substrates [e.g., Fast Red, BCIP/INT, available from (ABCAM, Cambridge, Mass.)]. Direct labeling can be achieved by covalently conjugating a label molecule to the polynucleotide (e.g., using solid-phase synthesis) or by incorporation via polymerization (e.g., using an in vitro transcription reaction or random-primed labeling). Indirect labeling can be achieved by covalently conjugating or incorporating to the polynucleotide a non-labeled tag molecule (e.g., Digoxigenin or biotin) and subsequently subjecting the polynucleotide to a labeled molecule (e.g., anti-Digoxigenin antibody or streptavidin) capable of specifically recognizing the non-labeled tag.

The above-described polynucleotides can be employed in a variety of RNA detection methods such as Northern blot analysis, reverse-transcribed PCR (RT-PCR) [e.g., a semi-quantitative RT-PCR, quantitative RT-PCR using e.g., the Light Cycler™ (Roche)], RNA in situ hybridization (RNA-ISH), in situ RT-PCR stain [e.g., as described in Nuovo G J, et al. 1993, Intracellular localization of polymerase chain reaction (PCR)-amplified hepatitis C cDNA. Am J Surg Pathol. 17: 683-90, and Komminoth P, et al. 1994, Evaluation of methods for hepatitis C virus detection in archival liver biopsies. Comparison of histology, immunohistochemistry, in situ hybridization, reverse transcriptase polymerase chain reaction (RT-PCR) and in situ RT-PCR. Pathol Res Pract., 190: 1017-25] and oligonucleotide microarray analysis [e.g., using the Affymetrix microarray (Affymetrix®, Santa Clara, Calif.)].

For detection of gene amplification, the present invention may utilize various DNA detection methods such as Southern blot analysis, PCR; quantitative PCR, real time PCR, QS-PCR and restriction fragment length polymorphism (RFLP).

According to the present teachings, single nucleotide polymorphisms (SNP) can also be identified in placental-derived microparticles using a variety of approaches suitable for identifying sequence alterations. One option is to determine the entire gene sequence of a PCR reaction product. Alternatively, a given segment of nucleic acid may be characterized on several other levels. At the lowest resolution, the size of the molecule can be determined by electrophoresis by comparison to a known standard run on the same gel. A more detailed picture of the molecule may be achieved by cleavage with combinations of restriction enzymes prior to electrophoresis, to allow construction of an ordered map. The presence of specific sequences within the fragment can be detected by hybridization of a labeled probe, or the precise nucleotide sequence can be determined by partial chemical degradation or by primer extension in the presence of chain-terminating nucleotide analogs.

The presence of a sequence alteration (e.g., SNP) in the fetal genes is typically determined using methods which involve the use of oligonucleotides that specifically hybridize with the nucleic acid sequence alterations in the fetal gene, such as those described hereinabove.

According to the present teachings, any known SNPs detection method may be employed, as for example, restriction fragment length polymorphism (RFLP), sequencing analysis, microsequencing analysis, solid-phase microsequencing, MALDI-TOF Mass Spectrometry, mismatch detection assays based on polymerases and ligases, LCR (ligase chain reaction), Gap LCR (GLCR), Ligase/Polymerase-mediated Genetic Bit Analysis™, hybridization assay methods, hybridization to oligonucleotide arrays, allele-specific oligonucleotides (ASOs), Denaturing/Temperature Gradient Gel Electrophoresis (DGGE/TGGE), Temperature Gradient Gel Electrophoresis” (TGGE), Single-Strand Conformation Polymorphism (SSCP), dideoxy fingerprinting (ddF), Pyrosequencing™ analysis, Acycloprime™ analysis and reverse dot-blot. Furthermore, integrated systems (e.g. multicomponent integrated systems) and microfluidic systems may be used to analyze sequence alterations

U.S. Pat. No. 5,451,503 provides several examples of oligonucleotide configurations which can be utilized to detect SNPs in template DNA or RNA.

As mentioned above, analyzing a characteristic of a fetus can also be effected by determining a level of a polypeptide in placental derived microparticles.

Thus, once placental derived microparticles are isolated, polypeptides are extracted using methods which are well known in the art (e.g. cell lysis techniques) and the presence and/or level of a specific polypeptide can be determined using, for example, specific antibodies via the formation of an immunocomplex [i.e., a complex formed between the fetal amino acid sequence present in the placental derived microparticles and the antibody].

The immunocomplex of the present invention can be formed at a variety of temperatures, salt concentration and pH values and those of skills in the art are capable of adjusting the conditions suitable for the formation of each immunocomplex.

The term “antibody” as used in this invention includes intact molecules as well as functional fragments thereof, such as Fab, F(ab)2, Fv or single domain molecules such as VH and VL to an epitope of an antigen. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; (5) Single chain antibody (“SCA”), a genetically engineered molecule containing the variable region of the light chain and the variable region of the heavy chain, linked by a suitable polypeptide linker as a genetically fused single chain molecule; and (6) Single domain antibodies are composed of a single VH or VL domains which exhibit sufficient affinity to the antigen.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

According to the method of this aspect of the present invention, detection of immunocomplex formation is indicative of a presence of a polypeptide within the placental derived microparticles. The presence of such a polypeptide may be indicative of a fetal characteristic or a genetic mutation, alternatively, lack of a polypeptide may indicate of a fetal characteristic or a genetic mutation. Various methods can be used to detect the formation of the immunocomplex of the present invention and those of skills in the art are capable of determining which method is suitable for analysis (described in further detail below).

The antibody used in the immunocomplex of the present invention can be labeled using methods known in the art. It will be appreciated that the labeled antibodies can be either primary antibodies (i.e., which bind to the specific polypeptide) or secondary antibodies (e.g., labeled goat anti rabbit antibodies, labeled mouse anti human antibody) which bind to the primary antibodies. The antibody can be directly conjugated to a label or can be conjugated to an enzyme.

Antibodies of the present invention can be fluorescently labeled (using a fluorescent dye conjugated to an antibody), radiolabeled (using radiolabeled e.g., ¹²⁵I, antibodies), or conjugated to an enzyme (e.g., horseradish peroxidase or alkaline phosphatase) and used along with a chromogenic substrate to produce a colorimetric reaction. The chromogenic substrates utilized by the enzyme-conjugated antibodies of the present invention include, but are not limited to, AEC, Fast red, ELF-97 substrate [2-(5′-chloro-2-phosphoryloxyphenyl)-6-chloro-4(3H)-quinazolinone], p-nitrophenyl phosphate (PNPP), phenolphthalein diphosphate, and ELF 39-phosphate, BCIP/INT, Vector Red (VR), salmon and magenta phosphate (Avivi C., et al., 1994, J. Histochem. Cytochem. 1994; 42: 551-4) for alkaline phosphatase enzyme and Nova Red, diaminobenzidine (DAB), Vector(R) SG substrate, luminol-based chemiluminescent substrate for the peroxidase enzyme. These enzymatic substrates are commercially available from Sigma (St Louis, Mo., USA), Molecular Probes Inc. (Eugene, Oreg., USA), Vector Laboratories Inc. (Burlingame, Calif., USA), Zymed Laboratories Inc. (San Francisco, Calif., USA), Dako Cytomation (Denmark).

Detection of the immunocomplex can be performed using fluorescence activated cell sorting (FACS), enzyme linked immunosorbent assay (ELISA), Western blot and radio-immunoassay (RIA) analyses, immunoprecipitation (IP) or by a molecular weight-based approach.

The present invention may also be used to analyze sequence alterations at the protein level.

Briefly, proteins are extracted from placental derived microparticles (as described hereinabove) and the presence of the specific polymorphs of the protein is detected. While chromatography and electrophoretic methods are preferably used to detect large variations in molecular weight, such as detection of a truncated protein generated by sequence alterations, immunodetection assays such as ELISA and Western blot analysis, immunohistochemistry, and the like, which may be effected using antibodies specific to a sequence alterations, are preferably used to detect point mutations and subtle changes in molecular weight.

As mentioned, analysis of fetal chromosomal aberrations may be carried out on genetic material obtained from isolated placental derived microparticles. Thus, the present teachings can be used to detect chromosomal abnormality such as chromosomal aneuploidy (i.e., complete and/or partial trisomy and/or monosomy), as well as chromosomal translocation, subtelomeric rearrangement, deletion, microdeletion, inversion and/or duplication (i.e., complete and/or partial chromosome duplication).

According to a specific embodiment, the chromosome comprises chromosome 1, 2, 3, 4, 5, 6, 7, 8, 9, 10, 11, 12, 13, 14, 15, 16, 17, 18, 19, 20, 21, 22, X or Y, or partial sequence thereof.

Isolating chromosomes from placental derived microparticles may be carried out as described herein above and may comprise fragmented chromosomes or intact chromosomes.

Analyzing fetal chromosomes may be carried out by any method known in the art, as for example, by fluorescent in situ hybridization (FISH), by primed in situ labeling (PRINS), by quantitative FISH (Q-FISH), by multicolor-banding (MCB), by chromosomal dyes such as orcein or single fluorescent dye (as previously described in U.S. Pat. No. 5,418,169), by QF-PCR (e.g. using QST*R plus kit as available for example from Elucigene) and/or by PCR (e.g. real time PCR).

According to a specific embodiment of the present invention, the present methods may be used to detect specific gene mutations using e.g. primers or probes specific for the mutation (e.g., FISH probes which are specific for a deletion).

Thus the present teachings may be used to detect chromosomal trisomies. Examples of chromosomal trisomies which may be detected by the present invention include, but are not limited to, trisomy 21 [using e.g., the LSI 21q22 orange labeled probe (Abbott cat no. 5J13-02)], trisomy 18 [using e.g., the CEP 18 green labeled probe (Abbott Cat No. 5J10-18); the CEP® 18 (D18Z1, alph-satellite) Spectrum Orange™ probe (Abbott Cat No. 5108-18)], trisomy 16 [using e.g., the CEP16 probe (Abbott Cat. No. 6137-17)], trisomy 13 [using e.g., the LSI® 13 SpectrumGreen™ probe (Abbott Cat. No. 5J14-18)], and the XXY, XYY, or XXX trisomies which can be detected using e.g., the CEP X green and Y orange probe (Abbott cat no. 5110-51); and/or the CEP® X SpectrumGreen™/CEP® Y (mu satellite) SpectrumOrange™ probe (Abbott Cat. No. 5J10-51).

Various other trisomies and partial trisomies can be detected in placental derived microparticles according to the present teachings. These include, but not limited to, partial trisomy 1q32-44 (Kimya Y et al., Prenat Diagn. 2002, 22:957-61), trisomy 9p with trisomy 10p (Hengstschlager M et al., Fetal Diagn Ther. 2002, 17:243-6), trisomy 4 mosaicism (Zaslav A L et al., Am J Med Genet. 2000, 95:381-4), trisomy 17p (De Pater J M et al., Genet Couns. 2000, 11:241-7), partial trisomy 4q26-qter (Petek E et al., Prenat Diagn. 2000, 20:349-52), trisomy 9 (Van den Berg C et al., Prenat. Diagn. 1997, 17:933-40), partial 2p trisomy (Siffroi J P et al., Prenat Diagn. 1994, 14:1097-9), partial trisomy 1q (DuPont B R et al., Am J Med Genet. 1994, 50:21-7) and/or partial trisomy 6p/monosomy 6q (Wauters J G et al., Clin Genet. 1993, 44:262-9).

The present teachings can also be used to detect several chromosomal monosomies such as monosomy X, monosomy 21, monosomy 22 [using e.g., the LSI 22 to (BCR) probe (Abbott, Cat. No. 5J17-24)], monosomy 16 (using e.g., the CEP 16 (D16Z3) Abbott, Cat. No. 6J36-17) and monosomy 15 [using e.g., the CEP 15 (D15Z4) probe (Abbott, Cat. No. 6136-15)].

The present invention can also be used to detect chromosomal abnormality in cases were one of the parents is a known carrier of such an abnormality. The present invention may also be used to detect chromosomal abnormalities (e.g. translocations and microdeletions) which are asymptomatic in the carrier parent, yet can cause major genetic diseases in the offspring. These include, but not limited to, mosaic for a small supernumerary marker chromosome (SMC) (Giardino D et al., Am J Med Genet. 2002, 111:319-23); t(11; 14) (p15; p13) translocation (Benzacken B et al., Prenat Diagn. 2001, 21:96-8); unbalanced translocation t(8; 11) (p23.2; p15.5) (Fert-Ferrer S et al., Prenat Diagn. 2000, 20:511-5); 11q23 microdeletion (Matsubara K, Yura K. Rinsho Ketsueki. 2004, 45:61-5); Smith-Magenis syndrome 17p11.2 deletion (Potocki L et al., Genet Med. 2003, 5:430-4); 22q13.3 deletion (Chen C P et al., Prenat Diagn. 2003, 23:504-8); Xp22.3. microdeletion (Enright F et al., Pediatr Dermatol. 2003, 20:153-7); 10p14 deletion (Bartsch O, et al., Am J Med Genet. 2003, 117A:1-5); 20p microdeletion (Laufer-Cahana A, Am J Med Genet. 2002, 112:190-3.), DiGeorge syndrome [del(22) (q11.2q11.23)], Williams syndrome [7q11.23 and 7q36 deletiops, Wouters C H, et al., Am J Med Genet. 2001, 102:261-5.]; 1p36 deletion (Zenker M, et al., Clin Dysmorphol. 2002, 11:43-8); 2p microdeletion (Dee S L et al., J Med Genet. 2001, 38:E32); neurofibromatosis type 1 (17q11.2 microdeletin, Jenne D E, et al., Am J Hum Genet. 2001, 69:516-27); Yq deletion (Toth A, et al., Prenat Diagn. 2001, 21:253-5); Wolf-Hirschhorn syndrome (WHS, 4p16.3 microdeletion, Rauch A et al., Am J Med Genet. 2001, 99:338-42); 1p36.2 microdeletion (Finelli P, Am J Med Genet. 2001, 99:308-13); 11q14 deletion (Coupry I et al., J Med Genet. 2001, 38:35-8); 19q13.2 microdeletion (Tentler D et al., J Med Genet. 2000, 37:128-31); Rubinstein-Taybi (16p13.3 microdeletion, Slough R I, et al., Am J Med Genet. 2000, 90:29-34); 7p21 microdeletion (Johnson D et al., Am J Hum Genet. 1998, 63:1282-93); Miller-Dieker syndrome (17p13.3), 17p11.2 deletion (Juyal R C et al., Am J Hum Genet. 1996, 58:998-1007); 2q37 microdeletion (Wilson L C et al., Am J Hum Genet. 1995, 56:400-7).

The present invention can also be used to detect inversions [e.g., inverted chromosome X (Lepretre, F. et al., Cytogenet. Genome Res. 2003. 101: 124-129; Xu, W. et al., Am. J. Med. Genet. 2003. 120A: 434-436), inverted chromosome 10 (Helszer, Z., et al., 2003. J. Appl. Genet. 44: 225-229)], cryptic subtelomeric chromosome rearrangements (Engels, H., et al., 2003. Eur. J. Hum. Genet. 11: 643-651; Bocian, E., et al., 2004. Med. Sci. Monit. 10: CR143-CR151) and/or duplications (Soler, A., et al., Prenat. Diagn. 2003. 23: 319-322).

The agents of the present invention which are described hereinabove may be included in a diagnostic kit preferably along with appropriate instructions for use and labels indicating FDA approval for use in prenatal analysis of a fetus. Thus, the kit may comprise a first agent (e.g. antibody such as anti-NDOG1 antibody) capable of specifically binding placental derived microparticles and another agent for analyzing at least one component (e.g. polynucleotide, chromosome or polypeptide) of the contents of the placental derived microparticles (e.g. oligonucleotide, probe, dye or an antibody). Optionally, the kit may also comprise additional agents for isolating nucleic acids or polypeptides from the placental derived microparticles. The kit may also include appropriate buffers and preservatives for improving the shelf-life of the kit.

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely; various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Selected Methods in Cellular Immunology”, W.H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Methods

Blood Collection and Preparation

Blood samples (20 ml) were collected from pregnant women and placed into blood collection tubes containing Sodium Citrate (1:10). Tubes were centrifuged twice at 1,500×g for 15 minutes in order to reach Poor-Platelet Plasma (PPP).

Human Villous Trophoblasts (HVT) Characterization Human villous trophoblasts (HVT) were labeled using mouse anti-human-trophoblast membranes NDOG1 (which characterized placental trophoblast cells, (Serotec, N.C., United States). Samples were incubated for 30 minutes at room temperature, washed, labeled with a secondary antibody (PE anti-mouse, Jackson ImmunoResearch Europe) for 30 minutes and rewashed. Samples were analyzed by FACS.

Placental Microparticle (MP) Characterization

Blood samples were obtained from pregnant women at 24 weeks of gestation. Blood cells were separated from plasma by centrifugation.

In order to specifically label the placental microparticles (trophoblast microparticles), PPP was labeled with NDOG1-PE or with PE mouse IgG Isotype control (Serotec, N.C., United States) by incubation for 30 minutes at room temperature. The labeled MPs were analyzed by fluorescence activated cell sorting (FACS). Standard 0.75 μm beads (BD Biosciences) were used to calibrate the MP size.

Separation of Placental Microparticles

Total microparticles (MPs) were isolated from the PPP (from about 10 ml samples) by high speed centrifugation. Next, the placenta specific MPs were separated from the total MP pellet by immunoprecipitation. First the MPs were labeled with anti-NDOG1 antibody and then the NDOG1-MPs complex was separated with anti-mouse magnetic beads (Bioadamt beads). The placental MPs pellet was then used for DNA, miRNA or mRNA purification.

MPs Nucleic Acid Extraction

DNA was isolated using DNA purification kit (EPICENTER) according to the user's manual. DNA quality and quantity was measured by Nanodrop.

In Vitro Trophoblast Culture and Isolation of MPs

Human villous trophoblasts (HVT), obtained from pregnancies at 20-24 weeks of gestation, were purchased from ScienCell (Carlsbad, Calif., USA). Cells were cultured in-vitro in a modified culture medium comprising 50% Trophoblast Medium with supplements (as provided by ScienCell), 22% DMEM, 22% F12, 4% fetal calf serum (FCS), 1% antibiotics (10,000 units/ml penicillin, 10 mg/ml streptomycin, 250 units/ml nyastatin), 0.0001% Amphotericin B, 3.5 U/ml heparin. Cells were plated in Nunclone plates or flasks, incubated at 37° C., 5% CO₂ and were used for experiments at passages 4-15.

In order to obtain microparticles, the cells were starved for 48 hours (the cells were grown in M-199 medium without serum) and the cells' supernatants were collected. Placental MPs were isolated from the supernatants by serial centrifugations. DNA was extracted from the placental MPs by DNA purification kit (EPICENTER).

Molecular QF PCR Analysis

Molecular analysis was carried out using QST*R plus kit (Elucigene), a highly multiplexed DNA fluorescent-based assay. The assay contained markers for chromosomes 13, 18, 21, X and Y and detected the most common viable autosomal trisomies and sex chromosome aneuploidies simultaneously in a single tube.

Molecular Gene Expression—PCR Analysis

Homozygosity for the T allele of the C677T polymorphism in the gene encoding the folate dependent enzyme 5,10-methylenetetrahydrofolate reductase (MTHFR) was examined. This mutation is a known risk factor for neural tube defects (previously described in e.g. BMJ 2004; 328:1535-1536).

677C->T mutation on the MTHFR gene were examined in DNA obtained from placental MPs from 20 weeks pregnant women by Real Time-PCR (Rotore-gene).

### Example 1

### The Antibody NDOG1 Specifically Binds Trophoblast Cells

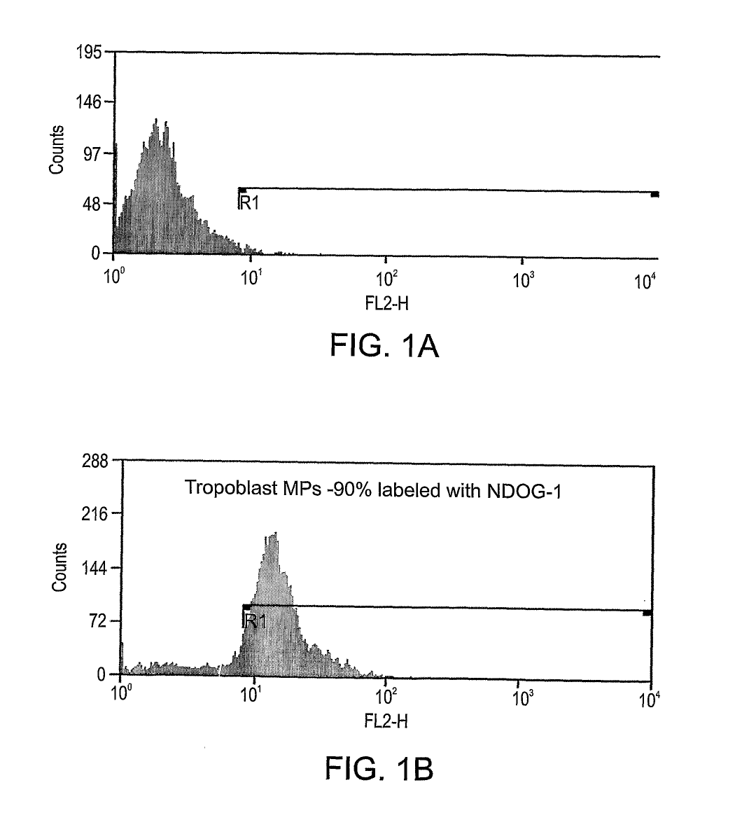

In order to demonstrate the specificity of NDOG1 to trophoblast cells, blood samples were obtained from 24 week pregnant women and placental human villous trophoblasts (HVT) present in the samples were specifically labeled using anti-NDOG1-PE. As shown in FIGS. 1A-B, approximately 90% of HVT expressed the NDOG1 antigen.

### Example 2

### Detection of NDOG1 Specific Microparticles in Pregnant Women

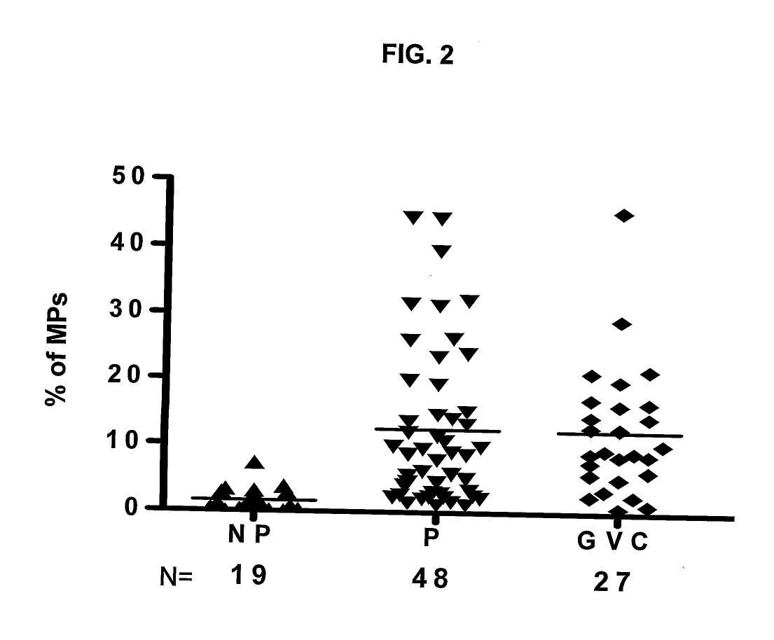

Microparticles isolated from poor platelet plasma of non-pregnant women (NP), healthy pregnant women (HP) and women with gestational vascular complications (GVC) were each labeled with anti-NDOG1 and evaluated by FACS. As illustrated in FIG. 2, both pregnancy groups had detectable levels of placental MPs compared to the non-pregnant group of women (p<0.0038).

### Example 3

### Elevation in Placental MP Levels in Early Stages of Pregnancy

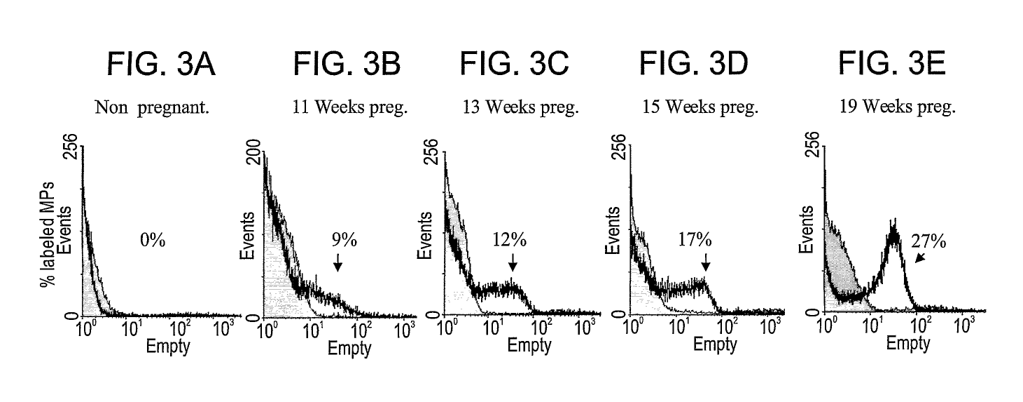

MPs were isolated from poor platelet plasma of non-pregnant women (NP) and from healthy pregnant women at different weeks of gestation (weeks 11, 13, 15 and 19 of pregnancy). As illustrated in FIG. 3, as the pregnancy progressed, more placental derived MPs were evident in the samples of healthy pregnant women.

### Example 4

### Placental MPs were Efficiently Separated from Total MPs

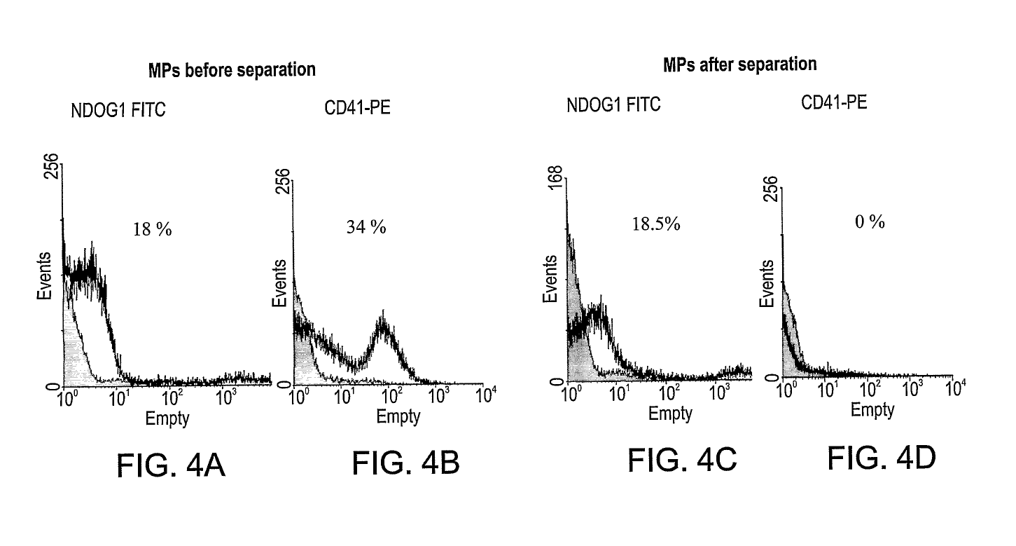

Placental MPs obtained from 15 week pregnant women were efficiently separated from total MPs using NDOG1 labeling and immunoprecipitation (as described in further detail hereinabove). As illustrated in FIGS. 4A-D, prior to separation, the total MPs comprised both placental specific MPs (labeled with anti-NDOG1, FIG. 4A) and maternal MPs (labeled with the anti-platelet marker CD41, FIG. 4B), however, after separation of the placental MPs, the MPs sample consisted of only placental MPs (FIG. 4C) and none of the MPs were labeled with maternal platelet marker, anti-CD41 (FIG. 4D).

### Example 5

### Determination of Microparticle Derived DNA Concentration and Quality

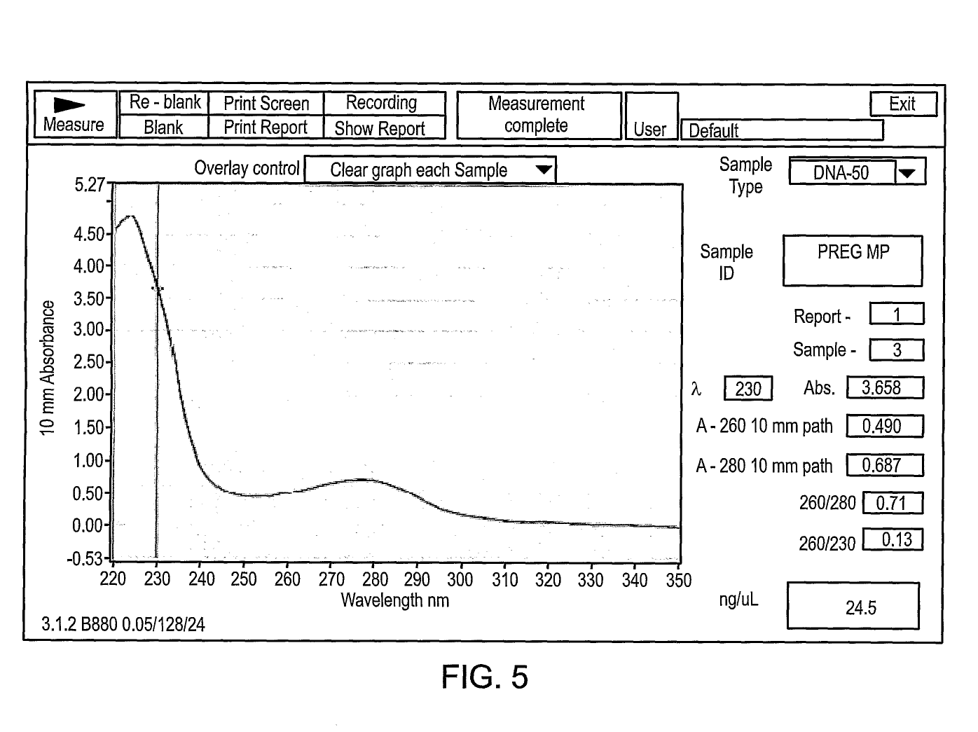

Placental MPs were isolated from poor platelet plasma (PPP) obtained from women at 19 weeks of gestation (as indicated in detail above). Next, DNA was extracted by purification kit (EPICENTER) and was evaluated for concentration and quality. As illustrated in FIG. 5, about 24 ng/μl DNA was obtained from the microparticles (from about 6 ml PPP).

### Example 6

### Genetic Profile of Trophoblast Derived Microparticles Using QF-PCR

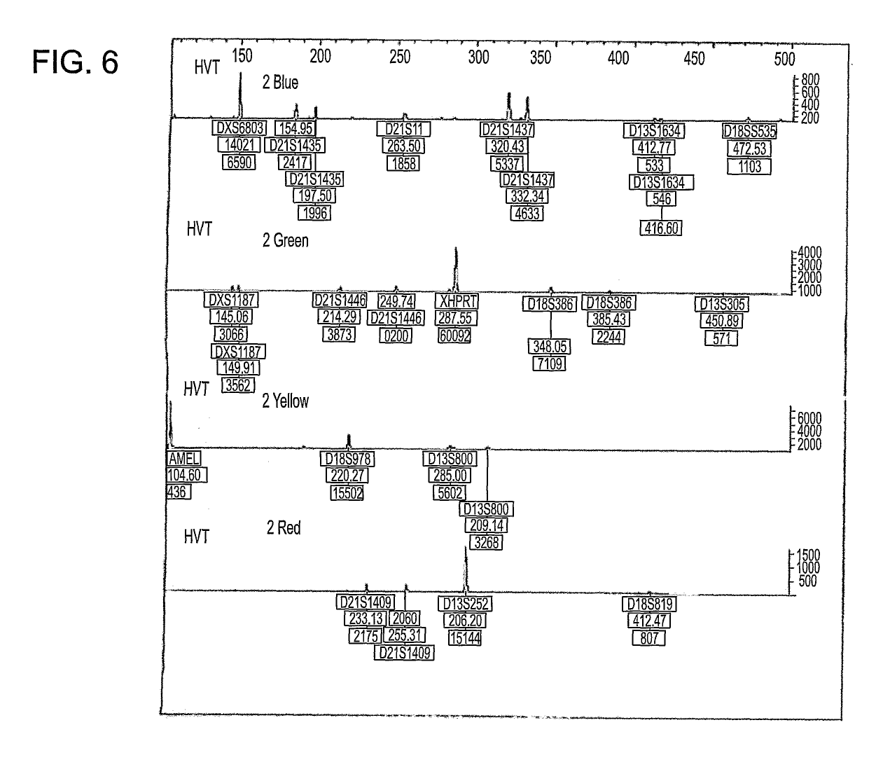

Trophoblast microparticles were separated from the supernatants of in-vitro grown trophoblasts (as indicated in detail hereinabove). DNA, was extracted from the trophoblast MPs and genetic profiling was carried out. As illustrated in FIG. 6, chromosomes 13, 18, 21, X and Y were detected.

### Example 7

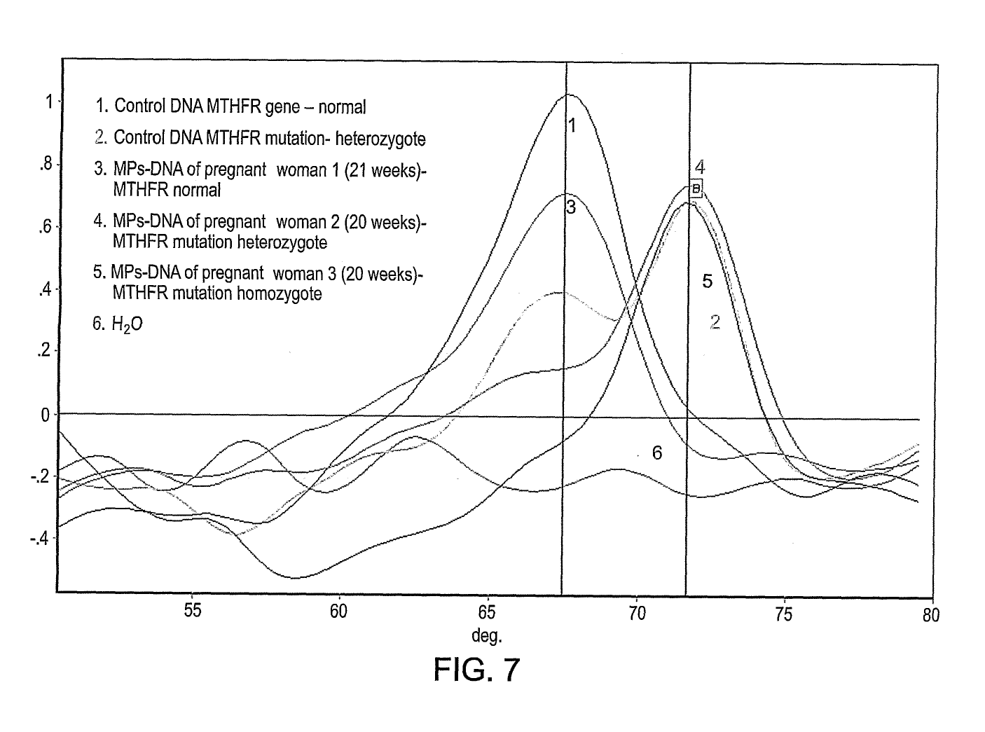

Placental-derived microparticles were separated from poor platelet plasma (PPP) of pregnant women. DNA was extracted from the placental MPs and genetic profiling for 5,10-methylenetetrahydrofolate reductase (MTHFR) polymorphism was carried out. As illustrated in FIG. 7, MTHFR mutations (heterozygote in placental-MPs of woman 2 and homozygote in placental-MPs of woman 3) were detected as well as MTHFR normal gene expression (in placental-MPs of woman 1).

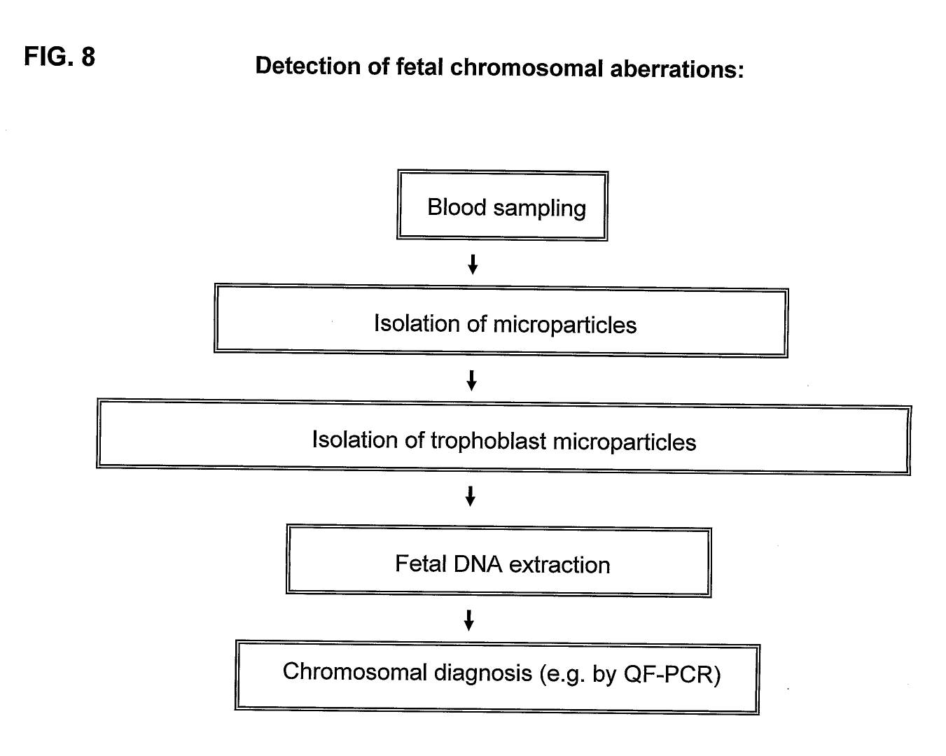

Taken together, the present results demonstrated that placental MPs may be specifically isolated from maternal blood and that DNA isolated from MPs is of good quality and quantity and can be further used for genetic evaluation, as for example, by PCR (for summary of the present invention see FIG. 8).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A prenatal method of analyzing a fetus, the method comprising:
(a) isolating placental derived microparticles; and
(b) analyzing at least one component of the contents of said placental derived microparticles, wherein said at least one component is indicative of a characteristic of the fetus.

2. (canceled)

3. A method of isolating placental derived microparticles from a blood sample obtained from a pregnant subject, the method comprising:
(a) contacting the blood sample with at least one agent which—specifically binds the placental derived microparticles and not to maternal microparticles under conditions that allow binding of said at least one agent to the placental derived microparticles; and
(b) isolating said placental derived microparticles, thereby isolating the placental derived microparticles from the blood sample.

4. (canceled)

5. The method of claim 3, wherein said isolating is effected by immunoprecipitation or FACS.

6. (canceled)

7. The method of claim 3, wherein said agent comprises an antibody.

8. (canceled)

9. (canceled)

10. The method of claim 3, wherein said agent binds a polypeptide selected from the group consisting of a human chorionic gonadotropin (HCG), a human Placental Lactogen (hPL), a NDOG1, a NDOG2, a NDOG5, a Trop-1 and a Trop-2 and any other antibodies against placental trophoblast markers including HLAG-MEM-G/1, HLAG-MEM-G/9, HLAG-MEM-G/11, HLAG-MEM-G233, anti human 5T4, anti trophoblast glycoprotein, IgG, and human PLAP (placental alkaline phosphatase).

11. (canceled)

12. The method of claim 1, wherein said at least one component comprises a nucleic acid.

13. The method of claim 1, wherein said at least one component comprises a polypeptide.

14. The method of claim 1, wherein said characteristic is a fetal disorder.

15. The method of claim 14, wherein said fetal disorder comprises a fetal chromosomal aberration.

16. (canceled)

17. The method of claim 14, wherein said fetal disorder comprises a fetal genetic mutation.

18. The method of claim 17, wherein said genetic mutation comprises polymorphism of the 5,10-methylenetetrahydrofolate reductase (MTHFR) gene.

19. The method of claim 1, wherein said characteristic is a sex of the fetus.

20. An isolated population of microparticles comprising at least 80% placental derived microparticles, obtained according to the method of claim 3.

21. A kit for prenatally analyzing a fetus, the kit comprising a packaging material packaging a first agent capable of specifically binding placental derived microparticles and a second agent for analyzing at least one component of the contents of said placental derived microparticles and instructions for use.

22. (canceled)

23. The kit of claim 21, wherein said first agent comprises an antibody.

24. The kit of claim 23, wherein said antibody comprises an anti-NDOG1 antibody or other anti-trophoblast antibodies as claimed in claim 10.

25. The kit of claim 21, wherein said at least one component is selected from the group consisting of a nucleic acid and a polypeptide.

26. The kit of claim 21, further comprising at least one agent for isolating nucleic acids from said placental derived microparticles.

27. The kit of claim 21, further comprising at least one agent for isolating polypeptides from said placental derived microparticles.

28. The kit of claim 21, wherein said second agent is selected from the group consisting of an oligonucleotide, a probe, an antibody and a dye.

29. (canceled)
